(19) United States
(12) Patent Application Publication
(10) Pub. No.: US 2021/0339086 A1
(43) Pub. Date: Nov. 4, 2021

(54) PRINTABLE ELECTRONIC GARMENT CONDUIT

(71) Applicant: Mad Apparel, Inc., Redwood City, CA (US)

(72) Inventors: James Artel Berg, San Francisco, CA (US); Wesley Groom, Redwood City, CA (US); Liang Yao, Redwood City, CA (US); Hamid Hameed Butt, Cupertino, CA (US); Anna Asnis, San Jose, CA (US); Dhananja Jayalath, Redwood City, CA (US)

(21) Appl. No.: 17/368,722

(22) Filed: Jul. 6, 2021

Related U.S. Application Data
(62) Division of application No. 16/198,769, filed on Nov. 22, 2018, now abandoned.
(60) Provisional application No. 62/590,143, filed on Nov. 22, 2017.

(51) Int. Cl. A63B 24/00; A41B 1/08; A63B 1/00

(52) U.S. Cl. CPC A63B 24/0062; A41D 1/005; A63B 1/00; A41B 1/08

(57) ABSTRACT

An athletic garment includes sensors at different locations of the garment. The sensors are electrically coupled to a processing unit and/or a power source via one or more conduits that are printed onto the garment. The conduits are designed for improved flexibility to accommodate stretching in the garment that occurs as a user wearing the garment performs an exercise and for improved durability to resist corrosion due to friction, sweat, or washing of the garment. In one embodiment, the conduits are designed for decreased stress concentrations at seams of the garment. In one embodiment, the conduits are designed to create a conductive pathway between different surfaces of the garment to electrically couple sensors on a first side (skin side) of the garment and a processing unit and/or a power source on a second side (outer side) of the garment.

### CROSS-REFERENCE TO RELATED APPLICATION

This application is a divisional application of U.S. patent application Ser. No. 16/198,769 filed on Nov. 22, 2018, which claims the benefit of U.S. Provisional Application No. 62/590,143, filed Nov. 22, 2017, all of which are incorporated by reference in their entirety.

### BACKGROUND

This description generally relates to sensor-equipped athletic garments, and specifically to conduit designs for electrically coupling sensors on the garment.

A garment can include sensors that record a variety of information about the human body. For example, electrocardiograph (ECG) electrodes can measure electrical signals from the skin of a person that are used to determine the person's heart rate. In addition, electromyography (EMG) electrodes can measure electrical activity generated by a person's muscles. Heart rate and muscle movement information may be useful for evaluating the person's physiological condition, for instance, while exercising. The sensors may be electrically coupled to a processing unit and/or a power source (e.g., a battery) via a plurality of conduits coupled to the garment.

As the garment may be designed to conform and stretch as a user wearing the garment performs an exercise, it is desirable for the conduits to have a certain amount of elasticity to adjust as the fabric of the garment stretches. However, the garment and the conduits are composed of materials having different elastic behavior and differing levels of durability. Also, seams of the garment may be areas of high stress concentrations as the fabric stretches, causing further strain on conduits that pass over the seams. In addition, the conduits may corrode over time due to friction or sweat during use of the garment and as the garment is washed and re-used. Moreover, some components of the exercise feedback system may be on an outside surface of the garment, such as the processing unit or the power source, while the sensors are positioned on the garment to make physical contact with the skin of a user wearing the garment and, thus, are on an inside surface of the garment. Accordingly, it is challenging to design garment conduits that are durable yet flexible and are able to create conductive pathways between different portions of the garment.

### SUMMARY

An athletic garment includes sensors that are located at different portions of the garment. The sensors are electrically coupled to a processing unit and/or a power source via one or more conduits.

In one embodiment, the garment comprises a first garment segment and a second garment segment. The first garment segment comprises a first edge, and the second garment segment comprises a second edge and is coupled to the first garment segment such that the first edge abuts the second edge, forming a seam along the first edge and the second edge. A buffer material is coupled to a surface of the first garment segment and the second garment segment such that the buffer material surrounds at least a first side of the seam. An electrical conduit is coupled to the surface of the first garment segment and the second garment segment such that a portion of the electrical conduit overlaps the buffer material and the seam.

In one embodiment, the garment comprises a first garment segment and a second garment segment. The first garment segment comprises a first electrical conduit on a first side of the first garment segment, where a portion of the first electrical conduit is exposed on a second side of the first garment segment through a hole within the first garment segment. The second garment segment comprises a second electrical conduit on a first side of the second garment segment, where a portion of the second electrical conduit is exposed on a second side of the second garment segment through a hole within the second garment segment. The hole within the first garment segment and the hole within the second garment segment are aligned such that the second side of the first garment segment and the second side of the second garment segment contact each other and where the first garment and the second garment are coupled together by a conductive material coupling the first electrical conduit to the second electrical conduit via the hole within the first garment segment and the hole within the second garment segment.

### BRIEF DESCRIPTIONS OF THE DRAWINGS

The disclosed embodiments have other advantages and features which will be more readily apparent from the following detailed description of the invention and the appended claims, when taken in conjunction with the accompanying drawings, in which:

FIG. 1 is a diagram of a sensor-equipped athletic garment 100, according to an embodiment.

FIG. 2A illustrates a conduit configuration for improving the elasticity of the conduit, according to an embodiment.

FIG. 2B illustrates a cross-sectional view of a conduit configuration for transferring conductivity between different sides of the garment 100.

FIG. 2C illustrates a cross-sectional view of a conduit configuration for transferring conductivity between different sides of the garment 100.

FIG. 2D illustrates a cross-sectional view of a conduit configuration for transferring conductivity between different sides of the garment 100.

FIG. 2E illustrates a cross-sectional view of a conduit configuration across a seam of the garment 100.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### I. GARMENT

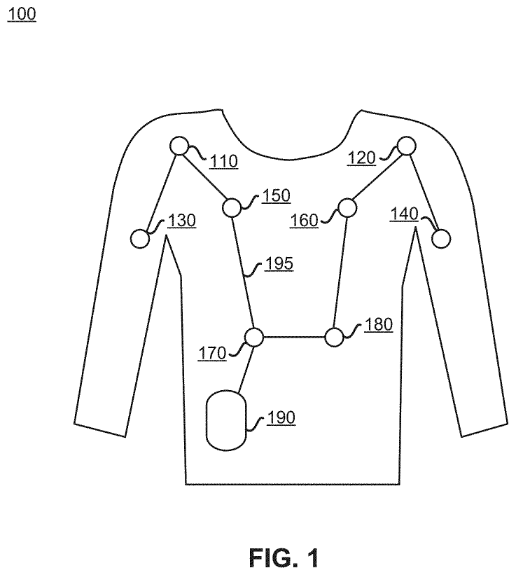

FIG. 1 is a diagram of a sensor-equipped athletic garment 100 according to one embodiment. The garment 100 may be part of an exercise feedback system that monitors the exercise performance of athletes. The garment 100 includes sensors that contact the skin of an athlete wearing the garment 100. For example, the sensors can be electrodes that measure electromyography (EMG) signals (electrical signals caused by muscle cells), also referred to as muscle activation data or physiological data, or electrocardiograph (ECG) signals (electrical signals caused by depolarization of the user's heart muscle in particular), also referred to as heart rate data. The sensors may also include other types of sensors such as accelerometers and gyroscopes (which generate motion data based on the athlete's movement), temperature sensors, pressure sensors, humidity sensors, etc. The sensors generate physiological data of the athlete based on the measured signals. The sensors are communicatively coupled to a processing unit 190. The processing unit 190 can aggregate and analyze the physiological data from the sensors. In some embodiments, the processing unit 190 may provide the physiological data to a client device (e.g., an athlete's device, a coach's device, etc.) or an exercise feedback system via a network.

In the embodiment shown in FIG. 1, the garment 100 includes eight sensors that record muscle activation data from the athlete's muscles nearby each sensor. For example, sensors 110 and 120 located on the right and left shoulder of the garment 100 can record muscle activation data of the athlete's deltoid muscles. Sensors 130 and 140 located on the right and left sleeves of the garment 100 can record muscle activation data of the athlete's triceps and/or bicep muscles. Sensors 150 and 160 located on the right and left chest of the garment 100 can record muscle activation data of the athlete's pectoral muscles. Sensors 170 and 180 located on the right and left abdomen of the garment 100 can record muscle activation data of the athlete's abdominal and oblique muscles. Though the garment 100 shown in FIG. 1 includes eight sensors and the processing unit 190, in other embodiments, the garment 100 can include any number of sensors or other types of components or electronics at any location or configuration within the garment 100.

It should be noted that while the garment 100 shown in FIG. 1 is a long sleeve shirt, the principles described herein apply equally to any garment, including but not limited to a short-sleeved shirt, a tank top, pants, shorts, or any other suitable garment. In embodiments where the garment 100 is a pant, sensors of the garment 100 can record physiological data from muscles on an athlete's lower body, e.g., quadriceps (also referred to herein as “quad” or “quads”), gluteus maximus (also referred to herein as “glute” or “glutes”), hamstrings, calves, and the like.

Generally, the garment 100 is made of fabric that includes, but is not limited to, cotton, cotton hybrids, polyester, polypropylene, nylon, spandex/LYCRA®, or any blend or combination thereof. These types of fabrics allow the garment 100 to conform to a user's body and to stretch as the user performs an exercise. Each sensor is embedded within the fabric and positioned such that the sensor can make physical contact with the skin of the user wearing the garment 100. Each sensor on the garment 100 is communicatively coupled to the processing unit 190 via conduits 195. Although the conduits 195 illustrated in FIG. 1 are straight lines, it should be noted that in other embodiments, the conduits 195 can have curved patterns, zigzag patterns, and the like. In the embodiment of FIG. 1, the conduits 195 can be made of printable conductive ink that is generally composed of conductive polymer, silver, carbon, silver chloride, gold, graphene, carbon nanotubes, or some combination thereof, can be conductive thread or wire, or can be made of any conductive material. The conduits 195 may be printed or embedded onto the fabric of the garment 100. In some embodiments, the conduits 195 may be printed onto an inlay material that is attached to the fabric of the garment 100. The inlay material provides a durable surface for the conduit to be printed onto and seals the conduits once printed, thereby protecting the printed conduits from sweat and other liquids. The conduits 195 may be on an inside surface of the garment 100 (closest to a user's skin surface), on an outside surface of the garment 100, or between various garment layers. It should be noted that for the remainder of the description herein, the conduits 195 are described as printed conduits for the purposes of simplicity, though it should be noted that the principles described herein apply to any suitable type of conduit described above. Printed conduits can improve the wearability of the garment 100 by reducing the need for physical wires connecting to each sensor and increasing the comfort and freedom for the user wearing the garment 100. Conduits may be printed with high precision printing machines. Using these machines allows the density of the conduits (i.e., the conduit area in a given area of the garment) to be increased while maintaining separation and avoiding electrical connection between adjacent printed conduits. In the embodiments in which conduits are printed onto inlay material, increasing conduit density may reduce the amount of area required for inlay material on the garment, which in turn increases the stretch of the garment (relative to a configuration having lower conduit density and thus an increased area of inlay material). Further, printing conduits may reduce manufacturing complexities associated with physical wires or thread and increase manufacturing throughput.

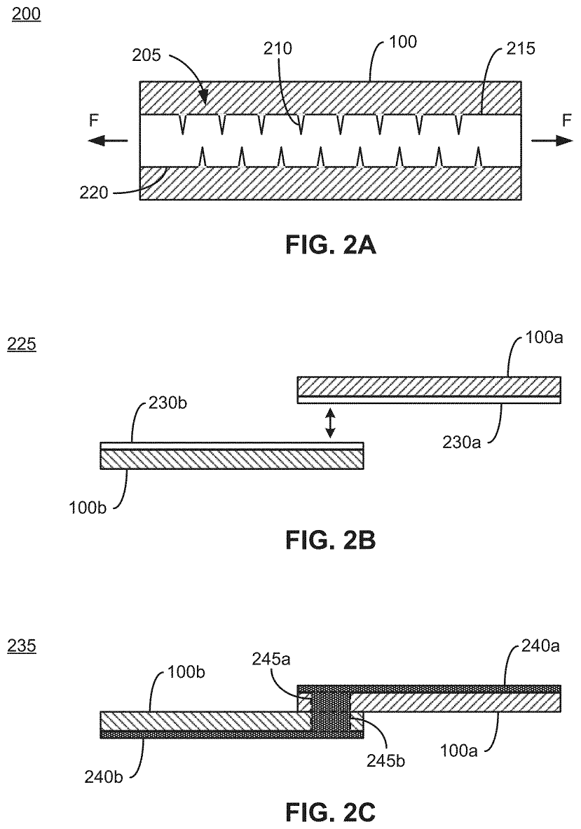

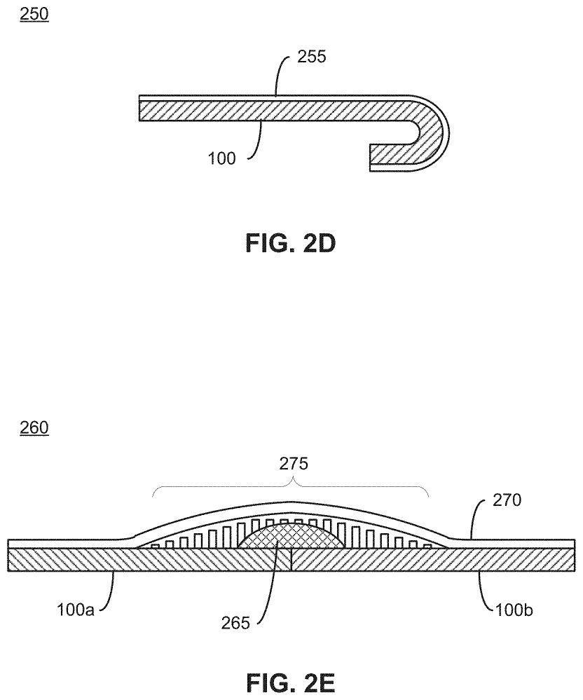

Given that the garment 100 is designed to conform and stretch as a user wearing the garment 100 performs an exercise, it is desirable for the conduits 195 to have a certain amount of elasticity to adjust as the fabric stretches. In addition, the conduits 195 may corrode over time due to friction or sweat during use of the garment 100 and as the garment 100 is washed and re-used. Since the garment 100 and the conduits 195 are composed of materials having different elastic behavior and differing levels of durability, the conduits 195 may be printed in various configurations to improve the elasticity and durability of the conduits 195. Moreover, some components of the exercise feedback system may be on an outside surface of the garment 100, such as the processing unit 190 or a power source (e.g., a battery). As previously described, the sensors are positioned on the garment 100 to make physical contact with the skin of a user wearing the garment 100 and, thus, are on an inside surface of the garment 100. To connect the different components, the conduits on the garment 100 may be arranged in different configurations to transfer conductivity between the different sides of the garment 100. The configurations are discussed in further detail with regards to FIGS. 2A-2E. The configurations described in further detail may be used alone or in some combination thereof.

### II. Conduit Configurations

FIG. 2A illustrates a conduit configuration 200 for improving the elasticity of the conduit, according to an embodiment. As illustrated in a top-down view in FIG. 2A, a conduit 205 is attached to the garment 100. The conduit 205 includes a plurality of notches 210 along a first edge 215 and a second edge 220 of the conduit 205. The notches 210 may be positioned at regular intervals (e.g., 0.5-15 millimeters) or at irregular intervals or apart from each other or at systematically varying regularity to design in a desired stretch at certain locations. As shown in FIG. 2A, the notches 210 along the first edge 215 are offset from the notches 210 along the second edge 220, forming an accordion-shaped conduit configuration. If the garment 100 is stretched in an axial direction such that a force F is applied at both ends of the conduit 205, the notches 210 may allow the conduit 205 to better respond and flex in response to the applied force. In the embodiment of FIG. 2A, the notches 210 are substantially triangular-shaped, but in other embodiments, the notches 210 may be square-shaped, rectangular, slits, elliptical, scalloped, or any other suitable shape. In some embodiments, the notches 210 may be located along only one edge or both edges down the length of the conduit 205. In some embodiments, the notches 210 may be located along a portion of the conduit 205 or along the entire length of the conduit 205. The conduit 205 may be printed onto an inlay material that is then attached to the garment 100, or the conduit 205 may be printed directly onto the garment 100. In embodiments in which the conduit 205 is printed onto an inlay material, the inlay material may be produced with the notch design as well to improve the flexibility of the overall stack-up of the inlay material and conduit.

FIG. 2B illustrates a cross-sectional view of a conduit configuration 225 for transferring conductivity between different sides or surfaces of the garment 100. As previously described, sensors of the garment 100 are located on an inside surface of the garment 100 to contact a skin surface of a user wearing the garment 100. Other components of the exercise feedback system, such as the processing unit 190 and/or a power source, may be located on an outside surface of the garment 100. FIG. 2B illustrates a first garment portion 100a and a second garment portion 110b. The garment portions 100a, 100b may be different pieces of fabric that are to be attached together to form the garment 100, or the garment portions 100a, 100b may be different portions of the same piece of fabric for the garment 100. In the embodiment of FIG. 2B, the garment portion 100a includes a conduit 230a on a first side and the garment portion 100b includes a conduit 230b on a first side. The conduits 230a, 230b may be printed onto an inlay material that is then attached to the garment portions 100a, 100b, or the conduits 230a, 230b may be printed directly onto the garment portions 100a, 100b.

In the embodiment of FIG. 2B, the conduit side of the first garment portion 100a may be configured as an inside surface of the garment 100 while the conduit side of the second garment portion 100b may be configured as an outside surface of the garment 100. It should be noted that the configuration may be switched in other embodiments. The first garment portion 100a and the second garment portion 100b may be attached to each other (e.g., sewn or adhered together) such that the conduit 230a contacts the conduit 230b. In this configuration, the conduit 230a and the conduit 230b transfer conductivity from an inside surface of the garment 100 (where the sensors may be) to an outside surface of the garment (where the processing unit 190 and/or the power source may be), thus allowing for the transmission of power, data, and/or electrical signals.

FIG. 2C illustrates a cross-sectional view of a conduit configuration 235 for transferring conductivity between different sides of the garment 100. Similar to the embodiment of FIG. 2B, FIG. 2C illustrates a first garment portion 100a including a conduit 240a on a first side and a second garment portion 100b including a conduit 240b on a first side. Each garment portion 100a, 100b includes a respective hole 245a, 245b that extends through the width of the garment portion 100a, 100b. In the embodiment of FIG. 2C, each hole 245a, 245b is filled with conductive ink to form part of the respective conduit 240a, 240b. The first garment portion 100a and the second garment portion 100b may be attached to each other (e.g., sewn or adhered together) such that the conduit 240a and the conduit 240b are on opposite surfaces of the garment 100 and the holes 245a, 245b are aligned such that the portion of the conduits 240a, 240b in the respective holes 245a, 245b contact each other and establish a conductive path. In this configuration, the portion of the conduits 240a, 240b in the respective holes 245a, 245b transfer conductivity from an inside surface of the garment 100 (where the sensors may be) to an outside surface of the garment (where the processing unit 190 and/or the power source may be), thus allowing for the transmission of power, data, and/or electrical signals.

In some embodiments, the holes 245a, 245b may be created in the garment portions 100a, 100b before the conduits 240a, 240b are printed onto the garment portions 100a, 100b. As the conduits 240a, 240b are printed, the conductive ink may collect and fill in the holes 245a, 245b. In other embodiments, an inlay material having respective holes may be attached to the garment portions 100a, 100b before the conduits 240a, 240b are printed onto the inlay material. In other embodiments, a hole may be created in a single piece of fabric, and a conduit may be printed on both sides of the piece of fabric over the hole such that the ink creates a connection over the edge of the hole. In some embodiments, the fabric of the garment 100 may be a porous material, such that the conductive ink may flow through the garment portions 100a, 100b rather than a hole in the garment portions 100a, 100b. In yet other embodiments, instead of conductive ink, a conductive pin, conductive grommet, conductive rivet, or other conductive material and/or structure is inserted through the conduit 240a, the holes 245a, 245b, and the conduit 240b, thereby establishing an electrical coupling between the conduits 240a, 240b. The conductive rivets may be designed to contact and/or compress against conductive material surrounding the holes 245a, 245b to provide a connection between the conduits 240a, 240b. In some embodiments, a conductive pin or a conductive grommet may include a sharp point or sharp feature for piercing through one or more layers of fabric and/or the conduits 240a, 240b to create a hole and establish a circuit between the conduits 240a, 240b. In this configuration, the holes 245a, 245b may be created by the conductive pin or grommet after the conduits 240a, 240b are printed onto the garment portions 100a, 100b. In some embodiments, the conductive pin, grommet, or rivet may be covered with conductive ink or conductive glue after it has been attached and connected to the conduits 240a, 240b.

FIG. 2D illustrates a cross-sectional view of a conduit configuration 250 for transferring conductivity between different sides of the garment 100. In the embodiment of FIG. 2D, a first side of the garment 100 includes a conduit 255. In some embodiments, the conduit 255 may be printed onto an inlay material that is attached to the garment 100. The garment 100 or the inlay material may be folded onto itself such that the conduit 255 is exposed on both an inside surface of the garment 100 and an outside surface of the garment 100. Once folded, the folded portions of the garment 100 may be sewn or adhered together. This configuration may streamline manufacturing of the garment 100 by eliminating the necessity to print conduits on multiple garment portions and then subsequently coupling the garment portions together while still producing the effect of having a portion of a conduit exposed on multiple garment surfaces.

FIG. 2E illustrates a cross-sectional view of a conduit configuration 260 across a seam 265 of the garment 100. In the embodiment of FIG. 2E, a first garment portion 100a and a second garment portion 100b are attached along respective edges, creating the seam 265. In some embodiments, the seam 265 may be created using thread, adhesives, or any other suitable material. Due to the thread or adhesive, seams may create height variations on a garment 100, which can create difficulties when printing conduits onto a garment 100. In addition, seams 265 are generally more rigid and less likely to flex or stretch than the rest of the garment 100, which can result in stress concentrations at the seam 265. This may negatively impact the durability of a conduit that crosses over a seam. FIG. 2E illustrates a conduit 270 crossing over the seam 265, which is surrounded by a material creating a height gradient 275 around the seam 265. The height gradient 275 smooths out the drastic change in height between the surface of the garment portions 100a, 100b and the seam 265. The height gradient 275 may be composed of a foam or any other suitable cushioning or smoothening material that is capable of stretching, creating a uniform surface area, and/or dispersing the stretching and shearing forces at the seam 265 across a larger surface area as the garment 100 is stretched. In other embodiments, a height gradient 275 may be created by printing several layers of the conduit 270 on top of each other rather than using additional material or some combination thereof may be used. In the embodiment of FIG. 2E, the height gradient 275 is printed in rows parallel to the seam 265, but in other embodiments, the height gradient 275 may be printed in rows perpendicular to the seam (e.g., rows going across the seam) to reduce the drastic height change that occurs at the seam 265.

Stress concentrations may also occur at the boundary at which a conduit connects to another component of the exercise feedback system (e.g., sensors, power source, processing unit) due to a sudden height change or sudden material change (i.e., sudden change in elastic properties). A height gradient 275 may also be used at these boundaries to lessen the sudden change and spread the stress concentration over a greater area.

In some embodiments, a redundancy pattern may be used to cross a conduit over a seam. In these embodiments, the conduit may split into an array of conduits that each cross the seam. In this configuration, if one conduit fails, there are remaining conduits to retain the conductive pathway. In addition, one or more of the conduits may be designed to take a majority of the strain earlier on such that those conduits fail before the other conduits in the array. In this way, the life cycle of the remaining conduits begins after the initial conduits fail.

In some embodiments, a conduit may be formed using several layers of different conduit materials (e.g., silver and carbon layers) that have different elastic parameters. Different conductive materials may have different stretch properties and different changes in resistance with stretch along different axes. Layering the different conduit materials may then reduce resistance changes with stretch and reduce noise coupling to the physiological signals with stretch. In this configuration, the different conduit materials may help each other absorb forces as the garment stretches. In addition, a conduit layer made of silver is more prone to corrosion, and a conduit layer of carbon may protect it and improve the overall durability of the conduit. In some embodiments, a conduit may include an additional material (e.g., zinc or other more corrosive material) that is layered on top of the silver and carbon layers to serve as a sacrificial layer and further protect the silver and carbon from corrosion (e.g., from sweat).

In some embodiments, a conduit may include a coating that improves its resistance to abrasion and corrosion. For example, an encapsulant polyurethane layer may further protect the conduit. As another example, a hydrophobic layer may repel water, which may decrease corrosion of the silver and carbon layers. In some embodiments, a conduit may include a layer that is conductive or semi- or electro-static that may shield the conduit from interference or noise. The conduit configurations described above may be used alone or in combination with each other.

### Additional Configuration Considerations

Throughout this specification, some embodiments have used the expression “coupled” along with its derivatives. The term “coupled” as used herein is not necessarily limited to two or more elements being in direct physical or electrical contact. Rather, the term “coupled” may also encompass two or more elements are not in direct contact with each other, but yet still co-operate or interact with each other, or are structured to provide a thermal conduction path between the elements.

Likewise, as used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Upon reading this disclosure, those of skilled in the art will appreciate still additional alternative structural and functional designs for camera controllers as disclosed from the principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed:

1. A garment comprising:
a first garment segment comprising a first edge;
a second garment segment comprising a second edge, wherein the second garment is coupled to the first garment segment by a seam along the first edge and the second edge;
a buffer material coupled to surfaces of the first garment segment and the second garment segment such that the buffer material overlaps the seam; and
an electrical conduit coupled to the surfaces of the first garment segment and the second garment segment and coupled the buffer material overlapping the seam such that the electrical conduit does not abut the seam.

2. The garment of claim 1, wherein the seam is created using thread or adhesives.

3. The garment of claim 1, wherein the buffer material is configured to minimize a change in height caused by a protrusion of the seam from the first garment segment and the second garment segment.

4. The garment of claim 3, wherein the buffer material comprises rows of electrical conduit on the surfaces of the first garment segment and the second garment, at least one row of electrical conduit having a different height from other rows of electrical conduit.

5. The garment of claim 1, wherein the buffer material comprises a material configured to create a uniform surface area across the seam.

6. The garment of claim 5, wherein the material is foam.

7. The garment of claim 1, wherein the buffer material is configured to disperse forces exerted on the seam caused by stretching of the garment when worn by a user.

8. The garment of claim 1, wherein the buffer material is oriented parallel or perpendicular to the seam.

9. The garment of claim 1, wherein the first garment segment and the second garment segment comprise parts of a same garment segment.

10. The garment of claim 1, wherein the first garment segment and the second garment segment comprise different garment segments.

11. The garment of claim 1, wherein the electrical conduit comprises a single conduit.

12. The garment of claim 1, wherein the electrical conduit comprises an array of conduits that each overlap the buffer material and the seam.

13. The garment of claim 12, wherein one or more of the conduits in the array of conduits are configured to receive more strain caused by stretching of the garment when worn by a user than other conduits in the array of conduits.

14. The garment of claim 1, wherein the electrical conduit comprises a plurality of layers of two or more conduits of different materials.

15. The garment of claim 1, wherein the electrical conduit comprises a protective coating.

16. A garment comprising:
a first surface of the garment and a second surface of the garment separated by a seam of the garment;
a buffer material surrounding the seam of the garment; and
an electrical conduit coupled to the first surface of the garment and the second surface of the garment without abutting the seam of the garment.

17. The garment of claim 16, wherein the buffer material is configured to minimize a change in height caused by a protrusion of the seam from the first surface and the second surface.

18. The garment of claim 16, wherein the buffer material is configured to disperse forces exerted on the seam caused by stretching of the garment when worn by a user.

19. The garment of claim 16, wherein the buffer material is oriented parallel or perpendicular to the seam.

20. The garment of claim 16, wherein the electrical conduit comprises a plurality of layers of two or more conduits of different materials.
